(19) United States
(12) Patent Application Publication
(10) Pub. No.: US 2009/0096986 A1
(43) Pub. Date: Apr. 16, 2009

(54) Unit and Method for Internally Guiding Vision in Fundus Cameras

(75) Inventors: Frank Teige, Jena (DE); Detlef Biernat, Jena (DE)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(73) Assignee: CARL ZEISS MEDITEC AG, Jena (DE)

(21) Appl. No.: 11/792,192

(22) PCT Filed: Jan. 18, 2006
(86) PCT No.: PCT/EP06/00380

§ 371 Date: Apr. 10, 2008

(30) Foreign Application Priority Data
Jan. 21, 2005 (DE) 10 2005 003 443.8

(51) Int. Cl. A61B 3/12; A61B 3/14

(52) U.S. Cl. 351/206; 351/211; 351/246

(57) ABSTRACT

The present invention is directed to an optoelectronic unit for directing the eye gaze of the patient during examination or documentation of the fundus of the eye. The solution according to the invention for internal eye gaze guidance in fundus cameras provides a spatial light modulator for generating the fixation mark comprising imaging optics by which the displayed fixation mark is imaged on the eye fundus by a semitransparent mirror arranged in an observation beam path and via a swivel mirror arranged in a documentation beam path. The spatial light modulator is connected by a control unit to an actuating element for positioning the fixation mark. The device, which is preferably provided for fundus cameras, can facilitate the diagnosis of diseases of the eye fundus in that the documented recordings can be exactly reproduced and therefore exactly compared. In particular, the tracking and presentation of the course of diseases can be improved in this way.

### CROSS-REFERENCE TO RELATED APPLICATIONS

This application claims priority of International Application No. PCT/EP2006/000380, filed Jan. 18, 2006 and German Application No. 10 2005 003 443.8, filed Jan. 21, 2005, the complete disclosures of which are hereby incorporated by reference.

### BACKGROUND OF THE INVENTION

a) Field of the Invention

The present invention is directed to an optoelectronic unit for directing the eye gaze of a patient during examination or documentation of the fundus of the eye. The device, which is preferably provided for fundus cameras, can facilitate the diagnosis of diseases of the eye fundus in that the documented recordings can be reproduced exactly and, therefore, exactly compared. In particular, the tracking and presentation of the course of diseases can be improved in this way.

b) Description of the Related Art

In the fundus cameras known from the prior art, the eye gaze is directed by external or internal fixation lights. In so doing, the patient is presented with an optically generated fixation point which is reflected into the observation beam path in different ways and accordingly made visible to the patient. This fixation point is generated in different ways.

U.S. Pat. Nos. 4,279,478 and 4,620,779 describe internal fixation devices in which an incandescent lamp coupled to a pinhole diaphragm serves as light source. The generated fixation points can be moved in the image plane by means of special operating controls that are connected to electromechanical adjusting devices.

In the internal fixation devices described in EP 1 138 256 A3 and U.S. Pat. No. 6,755,526 B2, point light sources are used to generate the fixation marks which can be moved and positioned in a corresponding manner by a joystick.

However, a drawback in these solutions in which the “point light source” is adjusted manually by special operating controls is that determined retinal areas can be presented in a reproducible manner only conditionally, and electronically assisted evaluation of the course of a disease is barely possible.

Another working principle for an internally generated fixation mark is described in U.S. Pat. No. 6,636,696. In this case, an LCD matrix is positioned in front of a surface radiator and individual matrix elements are controlled by special circuits by means of an operating control and can accordingly be made transparent. The fixation mark generated in this way is positioned by means of a joystick, whose movements are converted into electric signals and evaluated, by making other matrix elements transparent.

Although this overcomes the drawback of point light sources, that a reproducible presentation of certain retinal areas can only be realized conditionally, an electronically assisted evaluation of the course of a disease, while possible, can be carried out only with limited accuracy and at a high cost because of the working principle of the LCD display.

The LCDs, which are structured quite roughly, are operated in the L-NULL regime. The variability with which the fixation marks can be presented is limited owing to the application in transmitted light, with or without background illumination, or in incident light. Accordingly, multi-color and graphics-capable fixation marks are not possible with LCDs.

It is the object of the present invention to overcome these disadvantages of the prior art and to develop a solution by which a defined fixation point or a defined pattern of fixation points can be generated reproducibly at any time with a predetermined position and with sufficient positioning accuracy.

According to the invention, this object is met by a unit for internal gaze guidance in fundus cameras comprising a spatial light modulator which is provided for generating a fixation mark, imaging optics by which the fixation mark is imaged on the eye fundus by a semitransparent mirror arranged in an observation beam path and via a documentation beam path and wherein the light modulator is connected by a control unit to an actuating element for positioning the fixation mark.

In the solution according to the invention for internal eye gaze guidance in fundus cameras, a fixation mark generated by a spatial light modulator is imaged on the fundus by imaging optics, a semitransparent mirror arranged in the observation beam path, and a swivel mirror arranged in a documentation beam path, and the positioning of the fixation mark is carried out by means of an actuating element which is connected to the spatial light modulator by a control unit.

The proposed technical solution can be applied particularly in fundus cameras in which fixation marks are generated and correspondingly positioned for internal eye gaze guidance. However, it can be used in principle in other ophthalmologic instruments.

The invention will be described more fully in the following with reference to an embodiment example.

### BRIEF DESCRIPTION OF THE DRAWINGS

In the drawings:

FIG. 1 shows the basic construction of a fundus camera with the internal fixation device according to the invention; and

FIG. 2 shows the transmission behavior of the dichroic mirror that is used.

### DESCRIPTION OF THE PREFERRED EMBODIMENTS

In the unit, according to the invention, for internal eye gaze guidance in fundus cameras, a spatial light modulator is provided for generating the fixation mark. The spatial light modulator has imaging optics by which the fixation mark is imaged on the fundus via a semitransparent mirror arranged in an observation beam path and a swivel mirror arranged in a documentation beam path. The spatial light modulator is connected by a control unit to an actuating element for positioning the fixation mark.

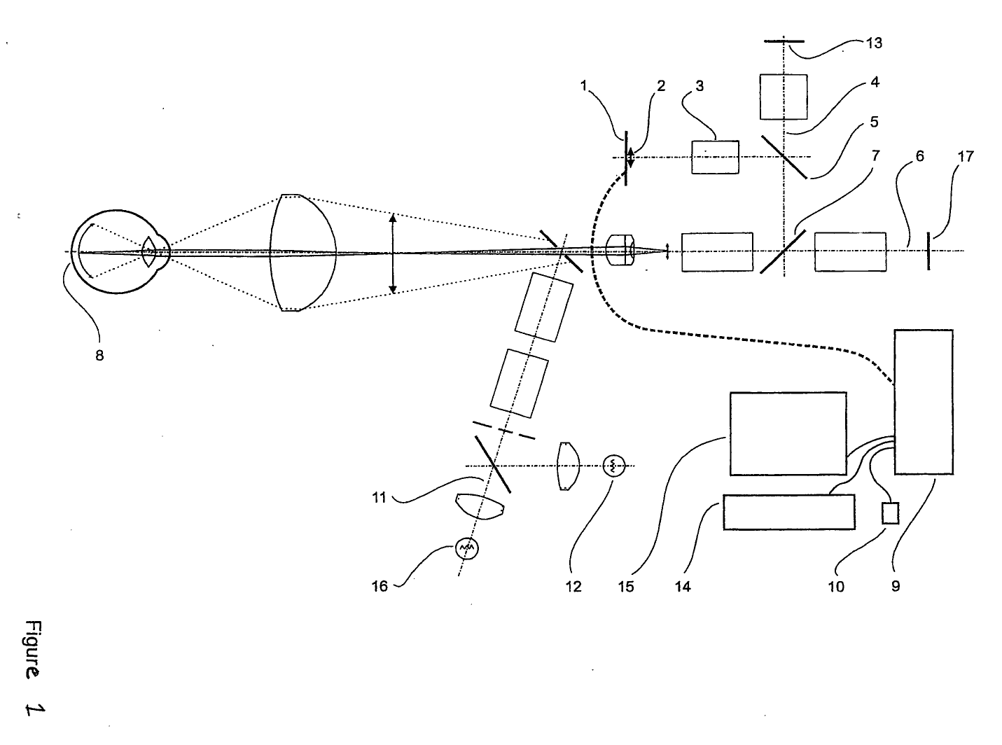

FIG. 1 shows the basic construction of a fundus camera with the internal fixation device according to the invention. A spatial light modulator in the form of high-resolution, graphics-capable LCDs 1 are provided for generating the fixation mark 2 and have imaging optics 3 by which the fixation mark 2 is imaged on the eye fundus 8 by a semitransparent, preferably dichroic, mirror 5 arranged in an observation beam path 4, a swivel mirror 7 arranged in a documentation beam path 6, and diverse optical elements. The spatial light modulator 1 is connected by a control unit 9 to an actuating element 10 for positioning the fixation mark 2.

Displays other than high-resolution, graphics-capable LCDs (liquid crystal displays), e.g., LCOS (liquid crystal on silicon), LED (light emitting diode), O-LED (organic light emitting diode), or DMD (digital micromirror device), can also be used as a spatial light modulator.

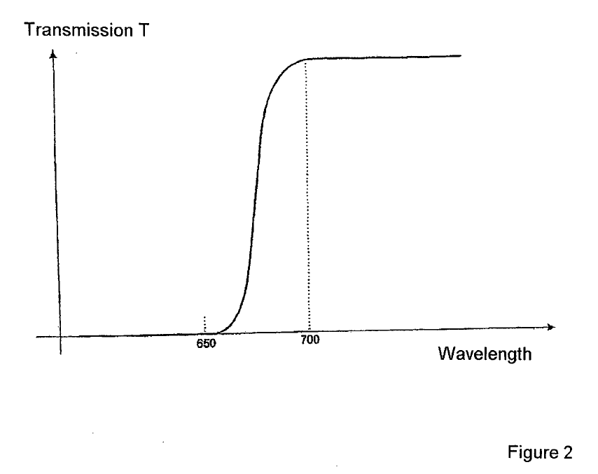

The dichroic mirror 5 arranged in the observation beam path 4 has the advantage that the light yield is as large as possible and stress on the patient is minimized as far as possible. FIG. 2 shows the transmission behavior of a dichroic mirror 5 to be used.

The infrared light for observation which is emitted by the light source 12 arranged in the illumination beam path is focused on the fundus 8 by different optical components, reflected by the fundus 8, and imaged on the CCD rangefinder camera 13 by the swivel mirror 7 and the dichroic mirror 5. The infrared illumination light is imaged in its entirety on the CCD rangefinder camera 13 corresponding to the transmission behavior of the dichroic mirror 5, while the fixation mark which is projected on the fundus 8 and lies in the visible spectral range is reflected.

The visible light for the image recording which is emitted by the strobe light source 16 arranged in the illumination beam path 11 is focused on the fundus 8 via different optical components, reflected by the fundus 8 and imaged on the CCD documentation camera 17 when the swivel mirror 7 is not located in the documentation beam path 6.

Since a PC is preferably used as control unit 9, the keyboard 14, a mouse, a trackball, a joystick, or the like, is used as an actuating element 10 for positioning the fixation mark 2 and is connected to the PC by a parallel, serial, USB or FireWire interface. It is also advantageous to use an additional monitor 15 for displaying the fundus image that is imaged in the observation beam path 4.

In order to examine and/or document certain fundus areas again at a later time, it is particularly advantageous to store data of the corresponding fixation mark such as, e.g., shape, color, intensity, coordinates, and the like, along with the corresponding fundus image. By activating this stored fixation mark, it is ensured that the patient gazes at a later time in the same direction so that the same fundus area is examined and/or documented. Changes are immediately visible by overlaying the images that were recorded at different times.

In the method according to the invention for internal eye gaze guidance in fundus cameras, a fixation mark 2 which is generated by a spatial light modulator in the form of high-resolution, graphics-capable LCDs 1 is imaged on the eye fundus 8 by imaging optics 3, a semitransparent mirror 5 arranged in an observation beam path 4, and a swivel mirror 7 arranged in a documentation beam path 6 and is accordingly visible to the patient. The positioning of the fixation mark 2 is carried out by means of an actuating element 10 which is connected by a control unit 9 to the spatial light modulator 1.

The coordinates of the pixels of the spatial light modulator 1 presenting the fixation mark 2 and, therefore, the position of the image of the displayed fixation mark 2 focused on the fundus 8 are changed by moving the actuating element 10. Since a PC is preferably used as control unit 9, a mouse, a trackball, a joystick, or the like connected to the PC by a parallel, serial, USB or FireWire interface is an obvious choice as an actuating element 10. Further, the PC serving as control unit 9 advantageously has a keyboard 14 for operation and a monitor 15 for displaying the fundus image that is imaged in the observation beam path 4.

When using a semitransparent mirror 5 in the observation beam path 4, the position or the movement of the fixation mark 1 which is positioned by means of the actuating element 10 and focused on the fundus 8 is displayed together with the image of the fundus on a monitor 15 connected to the control unit 9.

In contrast, when a dichroic mirror 5 is used in the observation beam path 4, the position or movement of the fixation mark 1 which is positioned by means of the actuating element 10 and focused on the fundus 8 is displayed in the form of a cursor corresponding to the fixation mark synchronously and, in a second plane, is superimposed on the image of the fundus and displayed on the monitor 15 connected to the control unit 9.

The use of a dichroic mirror 5 has the advantage that the light yield is as large as possible and stress on the patient is minimized as far as possible. FIG. 2 shows the transmission behavior of a dichroic mirror 5 to be used.

The infrared light for observation which is emitted by the light source 12 arranged in the illumination beam path 11 is focused on the fundus 8 by different optical components, reflected by the fundus 8, and imaged on the CCD rangefinder camera 13 by the swivel mirror 7 and the dichroic mirror 5. The infrared illumination light is imaged in its entirety on the CCD rangefinder camera 13 corresponding to the transmission behavior of the dichroic mirror 5, while the fixation mark which is reflected by the fundus 8 lies in the visible spectral range and is accordingly reflected by the dichroic mirror 5 and not shown in the viewing image.

The swivel mirror 7 is swiveled out of the documentation beam path 6 when the fundus 8 is photographed. The visible light which is emitted by the strobe light 16 arranged in the illumination beam path 11 is focused on the fundus 8 via different optical components, reflected by the fundus 8 and imaged on the CCD documentation camera 17. The internal fixation mark 1 is not shown on the recording.

In order to examine and/or document certain fundus areas again at a later time, it is particularly advantageous to store data of the corresponding fixation mark such as, e.g., shape, color, intensity, coordinates, and the like, along with the corresponding fundus image. By activating this stored fixation mark, it is ensured that the patient gazes at a later time in the same direction so that the same fundus area is examined and/or documented. Changes are immediately visible by overlaying the images that were recorded at different times. In order to correctly assess the course of diseases, it is necessary to obtain images or image sections of exactly the same fundus area and to overlay them.

The position of the fixation mark is entered simply and directly either by entering the corresponding coordinates by the keyboard or in that the fixation point is determined interactively by the examiner by clicking the mouse while observing the fundus of the patient. For repeat recordings, the coordinates of the fixation point can be read out of the stored data from the earlier recording and then entered manually or transferred directly from this recording by pressing a button.

Since the patient's eye to be examined should be fixated in its eye gaze direction by the generated fixation mark, the accuracy of the gaze direction is substantially determined by the size of the fixation mark and/or by the detail of its structures. The accuracy of the gaze direction is, in turn, very important for the reproducibility of the recordings. The fixation mark can be formed as an individual point or, for patients with defective vision, over a larger surface so that it can be more easily perceived by this group of patients. In addition, it is possible to vary the color of the displayed fixation mark. In order to direct the gaze of small children, it is even possible to generate colored, child-appropriate fixation marks in which certain details of the fixation mark can be designated as the target point so that an exact and reproducible guidance of eye gaze can be ensured for obtaining “coincident” images.

The solution according to the invention possesses an almost unlimited possibility for generating any desired shape of fixation mark. Any graphic shape that can be displayed on a PC monitor can also be used as a fixation mark in practice. For example, the often used “sighting rings” can be generated for patients suffering from macular degeneration,

The generated internal fixation mark is realized by the high-resolution, graphics-capable display with a sufficiently large number of image points in that every desired point of the display or groups of points can be activated in a software-controlled manner.

A substantial advantage of the method of guiding eye gaze according to the invention consists in that the reproducibility of digital recordings is appreciably improved by this method because the fixation mark can be generated again in a pixel-exact manner, that is, in the micrometer range. Accordingly, one or more recordings of the eye fundus that have been taken at different times can be made to coincide exactly. Therefore, by manipulating the images in a corresponding manner, it is easily possible for changes over time, i.e., the course of the disease, to be made visible in an appreciably clearer manner. The diagnosis and description of the course of a disease is substantially facilitated by corresponding image processing procedures such as differential image display or false color display.

Further, it is advantageous that the unit according to the invention and the method according to the invention for internal gaze guidance in fundus cameras can also be used for patients with defective vision because it is easily possible to generate fixation marks of different sizes. For such persons, the fixation mark is enlarged by means of clicking a mouse until it is visible for the patient. In this case, either the coordinates of all of the “partial points” taking part in the fixation mark can be stored, or the necessary data and details required for reproducing the shape and size of the specific fixation point can be stored in addition to the coordinates of the central “partial point”.

While the foregoing description and drawings represent the present invention, it will be obvious to those skilled in the art that various changes may be made therein without departing from the true spirit and scope of the present invention.

What is claimed is:

1-13. (canceled)

14. A unit for internal gaze guidance in fundus cameras, comprising:
a spatial light modulator being provided for generating a fixation mark;
imaging optics by which the fixation mark is imaged on the eye fundus by a semitransparent mirror arranged in an observation beam path and via a documentation beam path; and
said spatial light modulator being connected by a control unit to an actuating element for positioning the fixation mark.

15. The unit for internal gaze guidance according to claim 14, wherein a high-resolution, graphics-capable display is used as spatial light modulator.

16. The unit for internal gaze guidance according to claim 14, wherein the semitransparent mirror arranged in the observation beam path is preferably constructed as a dichroic mirror.

17. The unit for internal gaze guidance according to claim 14, wherein the actuating element for positioning the fixation mark can be a keyboard, mouse, trackball, joystick, or the like, which is connected to the PC serving as control unit by a parallel, serial, USB or FireWire interface.

18. The unit for internal gaze guidance according to claim 14, wherein the control unit additionally has a monitor for displaying the fundus image that is imaged in the observation beam path.

19. The unit for internal gaze guidance according to claim 14, wherein the control unit additionally has a device for storing the coordinates of the generated fixation mark and/or fundus image.

20. A method for internal gaze guidance in fundus cameras comprising the steps of:
generating a fixation mark by a spatial light modulator which is imaged on the eye fundus by imaging optics;
arranging a semitransparent mirror in an observation beam path and via a documentation beam path; and
positioning the fixation mark by an actuating element which is connected by a control unit to the spatial light modulator.

21. The method of claim 20 which utilizes a unit according to claim 14.

22. The method for internal gaze guidance according to claim 21, wherein a high-resolution, graphics-capable display is used as spatial light modulator.

23. The method for internal gaze guidance according to claim 21, wherein the coordinates of the pixels of the spatial light modulator presenting the fixation mark and, therefore, the position of the image of the displayed fixation mark focused on the fundus are changed by moving the actuating element, wherein a keyboard, a mouse, a trackball, a joystick, or the like, which is connected to the PC serving as control unit by a parallel, serial, USB or FireWire interface can be used as actuating element.

24. The method for internal gaze guidance according to claim 21, wherein the control unit additionally has a monitor for displaying the fundus image that is imaged in the observation beam path.

25. The method for internal gaze guidance according to claim 21, wherein when using a semitransparent mirror in the observation beam path, the movement of the fixation mark which is positioned by means of the actuating element and focused on the fundus is displayed together with the image of the fundus on a monitor connected to the control unit.

26. The method for internal gaze guidance according to claim 21, wherein when a dichroic, semitransparent mirror is used in the observation beam path, the movement of the fixation mark which is positioned by means of the actuating element and focused on the fundus is displayed, in addition to the image of the fundus, in the form of a cursor synchronously on a monitor connected to the control unit.

27. The method for internal gaze guidance according to claim 21, wherein the control unit stores the coordinates of the generated fixation mark for reexamining and/or redocumenting the same fundus area.
